(19) United States
(12) Patent Application Publication
(10) Pub. No.: US 2010/0292565 A1
(43) Pub. Date: Nov. 18, 2010

(54) MEDICAL IMAGING MEDICAL DEVICE NAVIGATION FROM AT LEAST TWO 2D PROJECTIONS FROM DIFFERENT ANGLES

(76) Inventors: ANDREAS MEYER, Bubenreuth (DE); MARCUS PFISTER, Bubenreuth (DE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/467,712

(22) Filed: May 18, 2009

(51) Int. Cl. A61B 5/05

(52) U.S. Cl. 600/424

(57) ABSTRACT

In a method or system for performing an image-assisted medical procedure involving placing a medical device in a 3D human subject, at least first and second 2D projected images are obtained of the 3D subject at a respective first angle and at a respective second angle, the 3D subject having at least one of the points selected from the group consisting of a start point and an end point for guidance of the medical device. At least one of the start point and the end point are identified in each of the first and second 2D images. 3D positions of at least one of the start point and the end point are calculated. At least one of these start and end point 3D positions are overlaid as a back-projection to a live 2D image. The procedure is performed utilizing the live 2D image with at least one of the start and end points as a guide to place the medical device. The medical device may comprise, for example, a wire or a needle.

### RELATED APPLICATIONS

The present application is related to the subject matter in pending U.S. patent application Ser. Nos. 12/023,906 filed Jan. 31, 2008 titled “Workflow to Enhance a Transjugular Intrahepatic Portosystemic Shunt Procedure”; 11/544,846 filed Oct. 5, 2006 titled “Integrating 3D Images Into Interventional Procedures”; and 11/900,261 filed Sep. 11, 2007 titled “Device Localization and Guidance”—all three of said U.S. patent applications being incorporated herein by reference.

### BACKGROUND

Registration of 3D medical imaging volumes or objects to 2D live images is a common technique to medical guide interventions performed on C-arm angiography systems (a C-arm is a rotatable C-shaped arm having an X-ray radiator and a detector with the patient being scanned being positioned therebetween as the arm rotates around the patient). These 3D volumes are usually acquired before the intervention (e.g. on a CT or MR) and then registered to the C-arm; or 2) acquired during the intervention (e.g. C-arm CT) and are thus automatically registered to the C-arm. Sometimes both approaches are not appropriate during interventions. Especially the rotation of the C-arm around the patient needed for 3D image acquisition may not be appropriate, e.g. if the room is very crowded, e.g. with anesthesia and additional devices. Also for some pediatric applications, where dose is an issue, one may want to avoid the acquisition of a full 3D run including between 120 to over 400 high dose X-ray projections.

### SUMMARY

In a method or system for performing an image-assisted medical procedure involving placing a medical device in a 3D human subject, at least first and second 2D projected images are obtained of the 3D subject at a respective first angle and at a respective second angle, the 3D subject having at least one of the points selected from the group consisting of a start point and an end point for guidance of the medical device. At least one of the start point and the end point are identified in each of the first and second 2D images. 3D positions of at least one of the start point and the end point are calculated. At least one of these start and end point 3D positions are overlaid as a back-projection to a live 2D image. The procedure is performed utilizing the live 2D image with at least one of the start and end points as a guide to place the medical device. The medical device may comprise, for example, a wire or a needle.

### BRIEF DESCRIPTION OF THE DRAWINGS

FIG. 1A shows obtaining 3D information about a certain object O by triangulation from at least two X-ray projections from different angles;

FIGS. 1B and 1C show obtaining of the two images from different angles achieved simultaneously or acquired sequentially;

FIGS. 2A, 2B and 2C illustrate the technique to guide a Transjugular Intrahepatic Porto Systemic Shunt (TIPS);

FIG. 3 shows a flow chart for the TIPS method steps;

FIGS. 4A, 4B and 4C show using the technique to guide Chronic Total Occlusion (CTO);

FIGS. 5A, 5B and 5C show using the technique to guide intracranial stentings (stroke);

FIGS. 6A, 6B and 6C show using the technique to guide stenting of Abdominal Aortic Aneurysms (AAA);

FIG. 7 is a flow diagram of steps of the technique for the above-described applications of localizing structures from 2D images from different angulations;

FIG. 8 is a flow diagram of a generic basic workflow for the technique;

FIG. 9 is a table showing needle or puncture guidance applications;

FIG. 10 is a table showing catheter guide wire or stent guidance applications;

FIG. 11 is a table showing neurosurgery and vascular surgery applications; and

FIG. 12 is a table showing spine procedures applications.

### DESCRIPTION OF THE PREFERRED EMBODIMENTS

For the purposes of promoting an understanding of the principles of the invention, reference will now be made to preferred embodiments illustrated in the drawings and specific language will be used to describe the same. It will nevertheless be understood that no limitation of the scope of the invention is thereby intended, and such alterations and further modifications in the illustrated devices and such further applications of the principles of the invention as illustrated as would normally occur to one skilled in the art to which the invention relates are included.

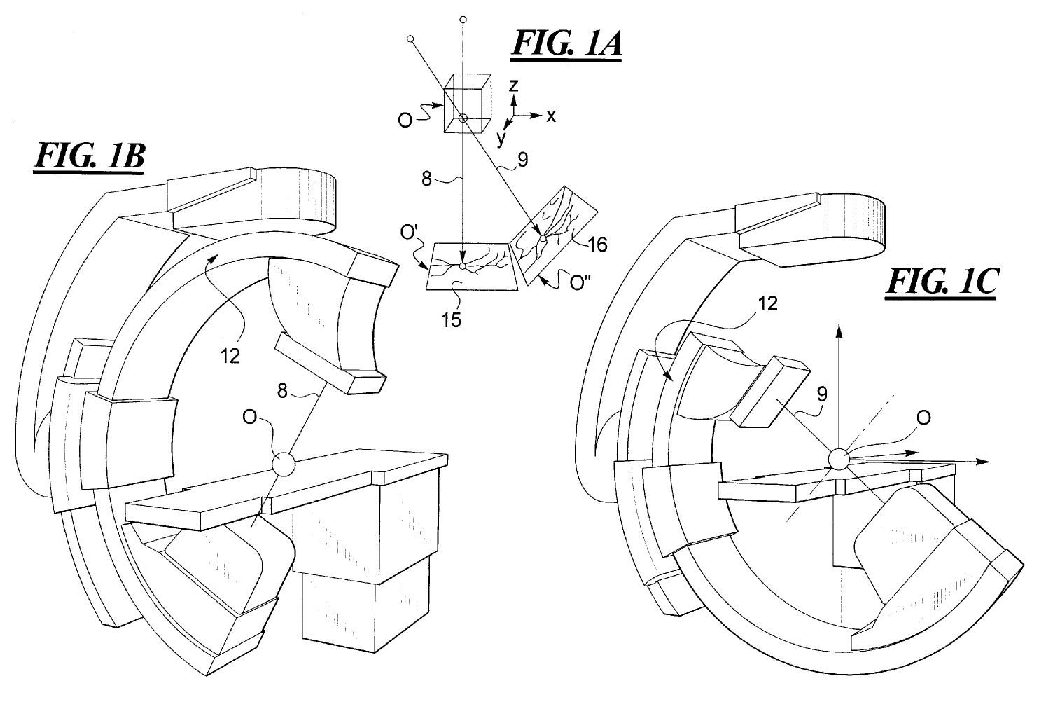

As shown in FIG. 1A, 3D information about a certain object O is obtained by the triangulation from at least two X-ray projections 8, 9 from different angles in which the projection O′ of object O at image 15 and projection O″ at image 16 is seen. In these projections, O′, O″ is localized (automatic, manual, e.g. by clicking on it, or semi-automatic), and the 3D position of O is computed by triangulation if the projection geometry is known. This is shown in FIG. 1A where the two images 15, 16 are achieved simultaneously, e.g. from a biplane system or acquired sequentially on a monoplane system. Additionally, a (semi-automatic/automatic) symbolic reconstruction of the region of interest may be performed and used as a 3D information (see e.g. German Patent Application 10 2008 057 702.2 filed Nov. 17, 2008 titled “Verfahren und Vorrichtung zur 3D-Visualisierung eines Gewebeabschnitts eines Gewebes” incorporated by reference herein).

Obtaining at least two 2D images from different angles to obtain a 3D information for localizing catheter and needle placement is described in U.S. application Ser. No. 11/900,261 incorporated herein by reference.

The above technique has been used and described to enhance TIPS (Transjugular Intrahepatic Porto Systemic Shunt) procedures see U.S. Ser. No. 12/023,900—or needle localization see U.S. Ser. No. 11/900,261—both incorporated herein by reference. In the following workflows, respective images to use are described to guide additional medical applications.

The preferred embodiments are used to guide different kinds of applications.

Just as is done for 2D3D registration (see U.S. Patent Publication 2008/014786 filed Oct. 5, 2006 titled “Integrating 3D Images Into Interventional Procedures” incorporated by reference herein), in the following, workflows are described to use the technique for different applications.

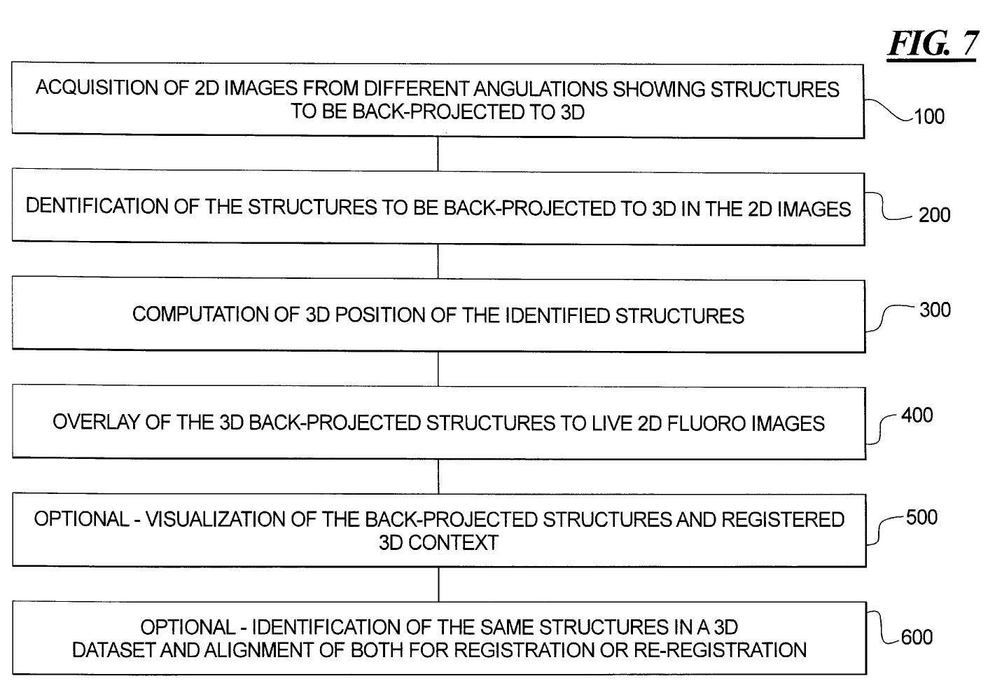

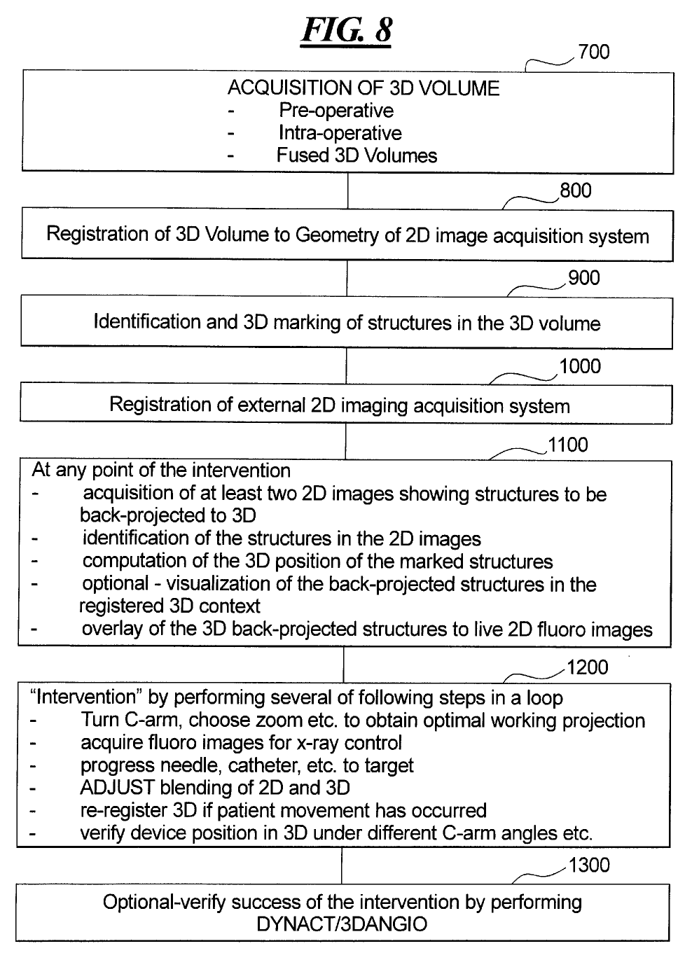

The technique of localizing structures from 2D images from different angulations is exploited, i.e. the steps performed are as follows as shown in the flow chart of FIG. 7:
acquisition of at least two 2D images from different angulations showing the structures to be back-projected to 3D (Block 100);
identification of these structures in the 2D images (Block 200);
computation of the 3D position of the identified structures (Block 300);
overlay of the 3D back-projected structures to live 2D fluoro images (Block 400).

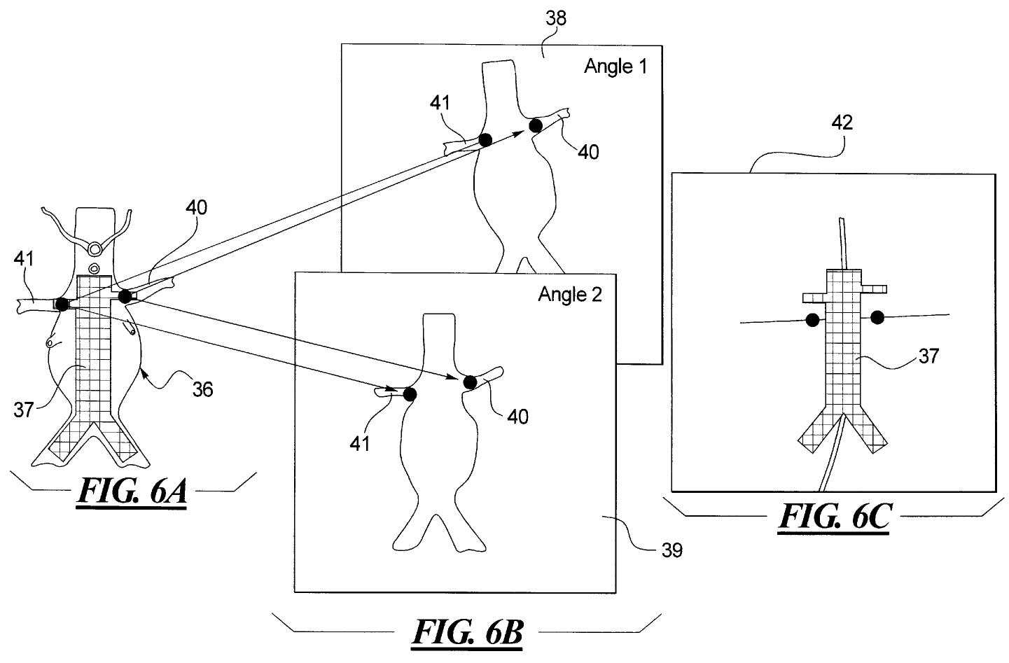

Optionally the following steps may be performed (FIG. 6):
visualization of the back-projected structures in the registered 3D context (Block 500); and
identification of the same structures in a 3D dataset and alignment of both for the purpose of registration or re-registration (Block 600).

The different workflows and technical realizations are described hereafter.

The advantage is the ability to guide medical procedures by 3D overlays which are easily created from just a few projection images, not an entire 3D run, which may not be achievable (or desired) in some clinical cases.

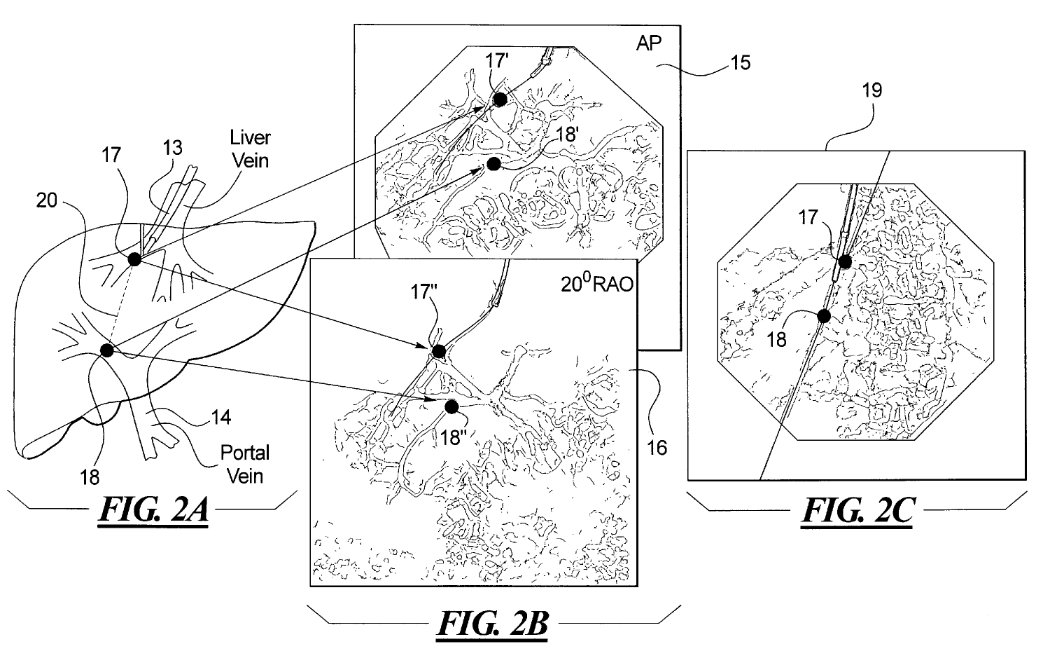

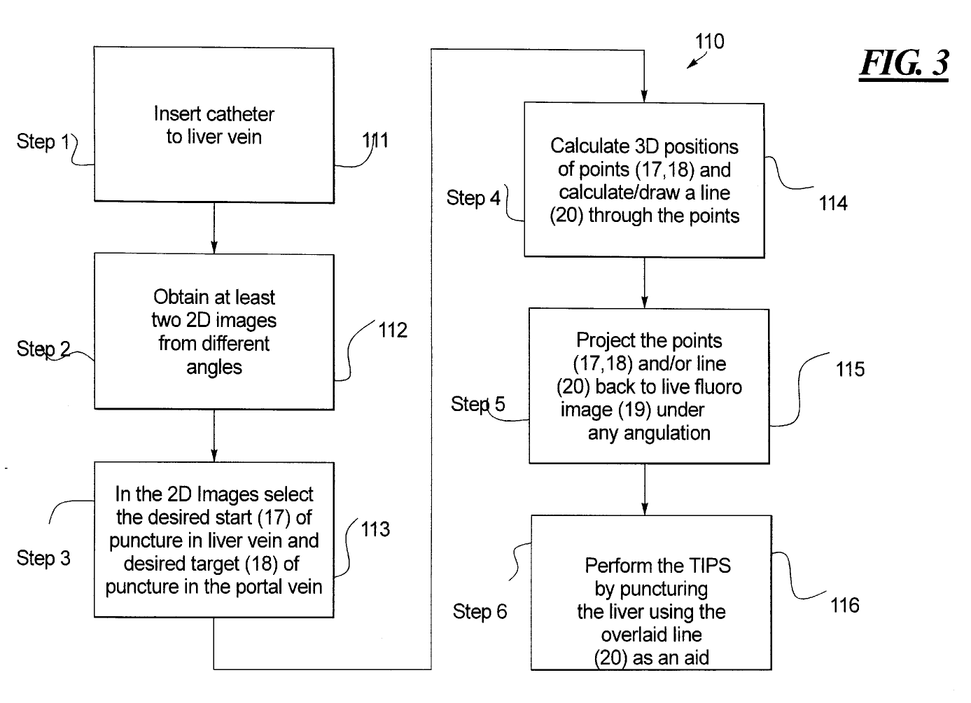

FIGS. 1A, B, C and FIGS. 2A, B show localization of an object O from two projections.

In FIG. 1A the projection O′ or O″ of an object O is shown in at least two X-ray projections 8, 9 from different angles and can be localized (automatic manual, e.g. by clicking on it, or semiautomatic), and the 3D position of O can be computed by triangulation if the projection geometry is known.

In FIGS. 1B and 1C, the two images 15, 16 are achieved simultaneously, e.g. from a C-arm Biplane System (two C-arms) or acquired sequentially on a Monoplane System (one C-arm 12). Additionally, a (semi-automatic/automatic) symbolic reconstruction of the region of interest may be performed and used as a 3D information (see e.g. German Patent Application 10 2008 057 702.2 incorporated herein by reference).

FIGS. 2A, B, C illustrate the technique to guide TIPS (see also German Patent Application 10 2008 057 702.2 incorporated herein by reference).

FIG. 2A shows a procedure in a TIPS, where a shunt between the liver vein 13 and the portal vein 14 is created.

FIG. 2B shows the 2D images 15, 16 to be used. Two protographies (image of the portal vein) (e.g. CO2-Wedge Angiographies) from different projections show the target 18 and preferably the start 17 of the puncture path.

FIG. 2C shows a navigation approach 19—a target and maybe a start of the puncture (pushing a medical device comprising a needle from liver vein 13 at 17 to the portal vein 14 at end point 18—dashed line 20) (Portal- and Liver Vein) are localized and back-projected to 3D. The information as shown in FIG. 2C is overlaid to live 2D as a puncture guidance.

As described in the previously mentioned U.S. Ser. No. 12/023,906 incorporated herein by reference, for the TIPS technique the steps shown in the flow chart of FIG. 3 are performed.

A software system is capable of back-projecting a point in 3D (see FIG. 3) by: 1) making use of this calibration; 2) having two 2D images 15, 16 from different angles of the structure to back-project; and 3) utilizing information as to where this structure is in the 2D image (e.g., by manually marking the structure). One desirable feature is to provide a 3D dataset of the liver via a preoperative procedure (by way of, e.g., a CT, MR, or other imaging procedure), or via an intraoperative procedure (by way of, e.g., C-arm CT, 3D Angio) which is registered to the C-Arm 12.

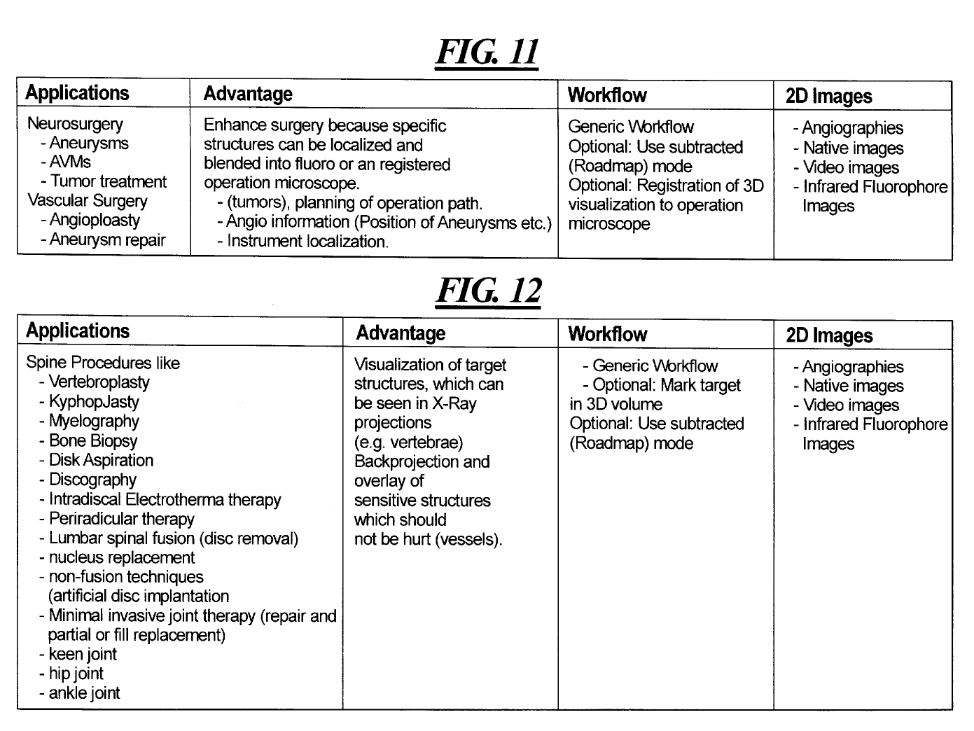

According to the embodiment of the method shown at 110 in FIG. 3 for a TIPS work flow illustrated in FIG. 3, in Step 1 at 111, a medical professional inserts a catheter up to the liver vein 13 (FIG. 2A). In Step 2 at 112, the medical professional then gets at least two 2D images 15, 16 (FIG. 2B) by performing the following steps: driving the C-Arm 12 to a first proper angulation in FIG. 1B; and getting an image 15 from that angle at 8 (e.g., a CO2 angiographic image), then driving the C-Arm 12 to a second proper angulation (FIG. 11C) at 9 by rotating it about the C-arm axis; and getting an image 16 from that angle at 9. The imaging is performed about imaging lines 8 and 9 respectively.

In Step 3 at 113, the medical professional (manually) localizes in the images 15, 16 (e.g., by selecting on them with a printing device) at least a desired start position 17′ (in the first image 15) and 17″ (in the second image 16) of the puncture in the liver vein 13, and a desired target 18′ in the first image 15) and 18″ (in the second image 16) of the puncture in the liver vein 20.

In Step 4 at 114 the system calculates the 3D positions of the two points 17, 18 via triangulation and draws a line 20 through the two points 17, 18. In Step 5 at 115, the system projects points 17, 18 and/or the line 20 back to the live fluoro images under any angulation. Finally, in Step 6 at 116, the medical professional performs the TIPS by puncturing the liver using the overlaid line 20 as an aid.

The tip of the medical device comprising the needle can be tracked in 3D, e.g., by the techniques described in U.S. Ser. No. 11/900,261 incorporated herein by reference, or with a position sensor, and the deviation of the tip from the planned path can be calculated and displayed. Also the tip of the needle can be displayed in 3D or within a registered 3D volume. The tip of the needle can be tracked in (one or more) 2D images with a position sensor, or automatically using a proper image processing algorithm.

In Step 2 at 112 in FIG. 3, different types of images can be imagined, such as native X-ray images, iodine contrast enhanced images from the liver and portal system (e.g., by inserting the catheter in the liver artery) as long as the structures to be localized (especially start and end of the puncture line) can be seen in them.

In Step 3 at 113 in FIG. 3, an arbitrary number of intermediate points can be marked and displayed. This could lead to a curved puncture line. In this step, as a puncture start, also the tip of the needle can be marked as a starting point.

Thus, according to these embodiments, a calculation and display of a puncture path is determined in 3D, and is potentially projected to a registered 3D volume and/or back-projected to the 2D live fluoro images by marking a start and end of the puncture path, e.g., by at least two images from different angulations (such as via CO2 angiographic images). This system and workflow can be used for any application which can make use of this feature of guiding a device through a volume (medical or not) by the described features.

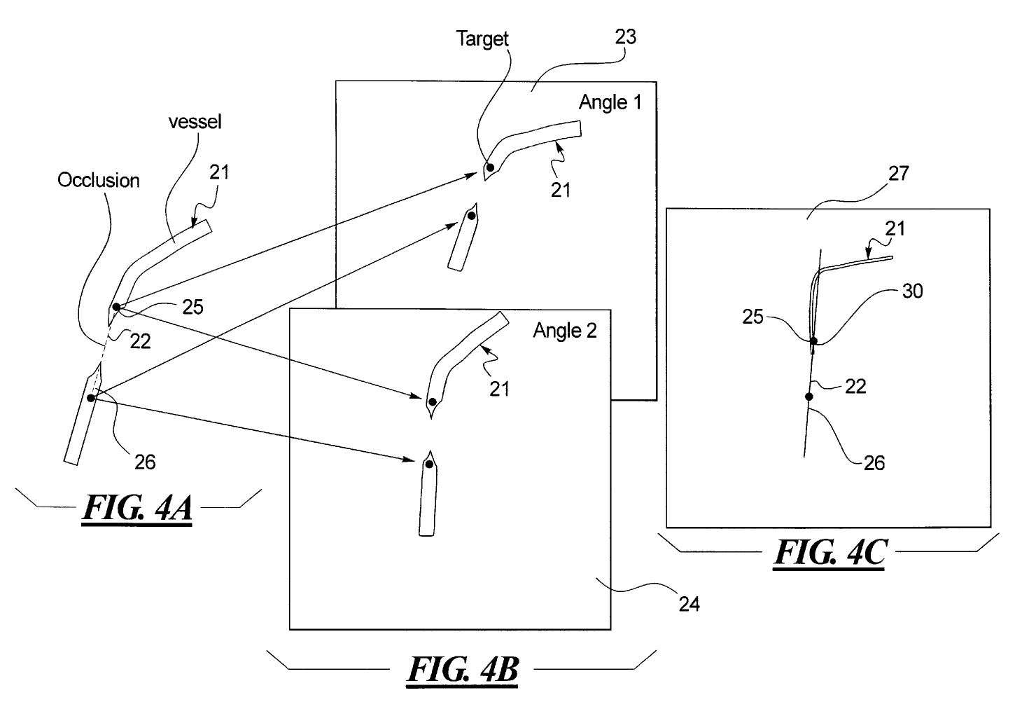

FIGS. 4A, B, C show using the technique to guide CTO (Chronic Total Occlusion-completely blocked blood vessel or coronary 21).

FIG. 4A shows a procedure wherein in a CTO procedure, an occluded coronary 21 is re-opened by pushing a medical device comprising a wire through a stenosis 22 (occluded area).

FIG. 4B shows 2D images 23, 24 to be used wherein two angiographies from different projections showing the start 25 and end 26 (e.g. through a retrograde filling of the vessel) of the stenosis 22.

FIG. 4C shows a navigation approach 27 wherein the start 25 and the end 26 of the stenosis 22 are localized and the 3D position is back-projected. The information is overlaid to live 2D as a guidance for the re-opening of the coronary 21.

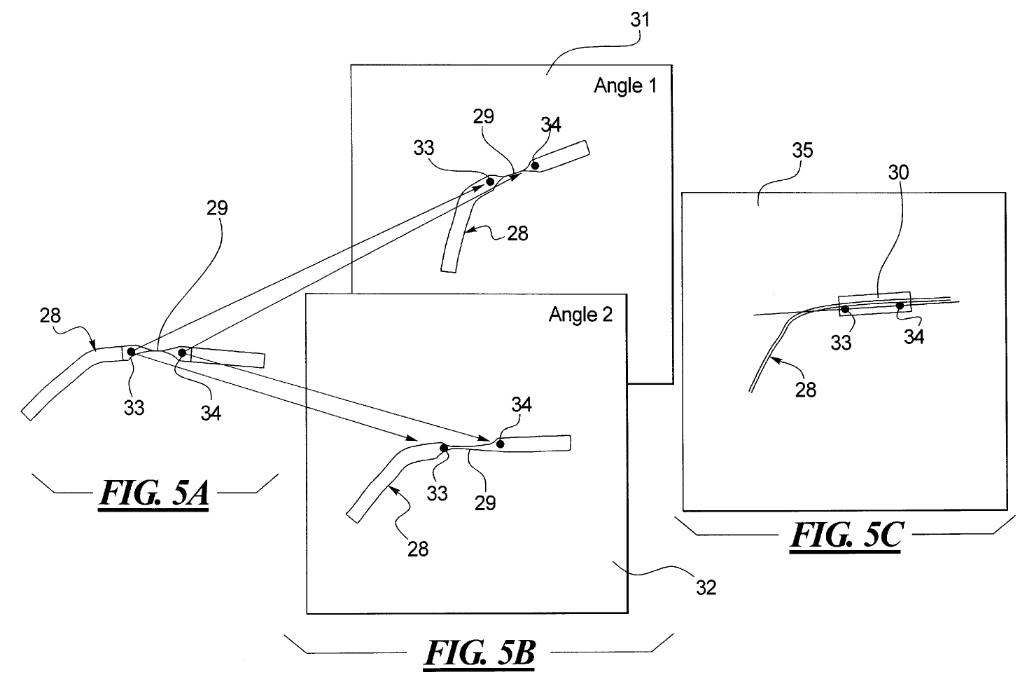

FIGS. 5A, B, C show using the technique to guide intracranial stentings (stroke).

FIG. 5A shows a procedure, e.g. because of a stroke, a (partly) occluded artery 28 in the brain is re-opened by pushing a medical device comprising a wire through a stenosis 29 and placing a stent at 30.

FIG. 5B shows 2D images 31, 32 to be used wherein two angiographies from different projections show the start 33 and the end 34 of the stenosis 29.

FIG. 5C shows a navigation approach wherein the start 33 and the end 34 of the stenosis 29 are localized and the 3D position 35 is back-projected. The information is overlaid to live 2D as a guidance for passing the artery and placing the stent at 30. Also information about the stent at 30 (e.g. length) can be obtained.

FIGS. 6A, B, C show using the technique to guide stentings of an abdominal aneurysm 36 (AAA).

FIG. 6A shows a procedure wherein the aortic aneurysm 36 is “repaired” pushing a medical device comprising a wire through the aneurysm 36 and placing a stent 37 to prevent it from rupturing.

FIG. 6B shows 2D images 38, 39 to be used wherein two angiographies from different projections show the renal artery branches 40, 41.

FIG. 6C shows a navigation approach 42 wherein the renal artery branches 40, 41 are localized and the 3D position is back-projected. The information is overlaid to live 2D as a guidance for placing the stent 37, so that it does not occlude these branches 40, 41.

Various possible embodiments, uses of the embodiments, and enhancements of the embodiments will now be described.

Different types of 2D images can be used to identify the structures to be back-projected to 3D. Those types are as follows:
subtracted angiographies (e.g. contrasted with iodine or CO2);
native contrasted images (e.g. contrasted with iodine or CO2);
native images (acquisitions or fluoroscopic images);
images taken by a 2D gamma camera;
2D ultrasound images;
2D optical images (e.g. by infrared images, e.g. enhanced by a fluorescent contrast agent;
2D video images (e.g. for surgery); and
combinations of the images described above.

An identification of points or structures to be back-projected in the 2D images is performed as follows. The structures seen in the 2D images from which a 3D position is computed (back-projected) are identified by several methods:
manual (by “clicking” on the structure);
automatic (image processing or segmentation); and
semiautomatic (e.g. by automatically segmenting a structure in 2D on which the user has clicked first).

Back-projected 3D information are the following:
points or graphic information (e.g. the point(s) which were clicked on, see e.g. German Patent Application 10 2008 057 702.2 incorporated herein by reference);
connecting lines between two or several points, see e.g. U.S. Ser. No. 12/023,906 incorporated herein by reference;
spline or other smoothing curve between three or more points;
symbolic reconstruction (e.g. of segmented regions (vessels) from the 2D images, see e.g. German Patent Application 10 2008 057 702.2 incorporated herein reference); and
combinations of the above.

3D reference frames in which the points or structures can be back-projected are as follows.

The structures seen in the 2D images from which a 3D position is computed (back-projected) are displayed in 3D context (i.e. the space to which they are projected. Possibilities are:
in 3D volumes (CT/MR/DynaCT) registered to the C-Arm (respectively the system on which the 2D images were acquired);
in anatomical or abstract phantoms (see German Patent Application 10 2008 054 298.9 filed Nov. 13, 2008 titled “Verfahren und Vorrichtung zur 3D-Visualisierung eines Eingriffspfades eines medizinischen Instrumentes, eines medizinischen Instrumentes und/oder einer bestimmten Gewebestruktur eines Patienten” incorporated herein by reference); and
into “Black Space”, i.e. no reference frame at all.

Additional features to be overlaid to live 2D images are as follows. If the structures are back-projected to a registered 3D, also structures in the 3D are marked to be overlaid to 3D. E.g. a needle tip is marked in two images from different angles and a tumor is marked directly in the registered 3D. The 3D then shows both markings (in 3D) and both are overlaid to live 2D fluoro.

Methods to update the alignment of the 3D structures and live 2D images are as follows. The structures back-projected in 3D (or other structures marked directly in 3D) are overlaid to 2D live fluoro images to guide the interventions. The following describes how the alignment can be maintained during the procedure.

A dynamic update of registration with a motion correction involves the update of the registration of the back projection of the marked structures with the live fluoro image, especially if patient motion occurs. The registration can be updated based on:
feature tracking (e.g. catheters, landmarks, diaphragm which move with breathing);
ECG triggering; and
respiratory tracking/control.

A dynamic update of registration with C-arm movement involves the update of the registration of the back projection of the marked structures with the live fluoro image especially if e.g. changes in X-ray system parameters, e.g. C-arm-movement, patient table movement etc. occur. The update is based on calibration information (i.e. the alignment is updated based on the knowledge of the current projection parameters).

For use of the back-projected structures, the structures marked in the 2D image are also identified and marked in a registered 3D volume. After this, they are aligned to re-register 2D and 3D.

A possible generic workflow with reference to the flow diagram of FIG. 8 for the preferred embodiment is as follows. For a basic workflow (See FIG. 8):

1. acquisition of 3D Volume (Block 700)
pre-operative (CT, MR, PET, SPECT . . . )
intra-operative (C-arm CT, 3D US), and
also fused 3D Volumes (such as CT+PET) to be used as the 3D volume;

2. registration of the 3D volume to the geometry of the 2D image acquisition system (e.g. the C-arm, gamma camera, infrared imager etc.) (Block 800);

3. identification and 3D marking of structures (e.g. a tumor) in the 3D volume (Block 900);

4. a registration of external 2D imaging acquisition system (e.g. gamma camera, infrared imager etc.) to the C-arm (Block 1000),

5. at any point of the intervention (also described in the flow diagram of previously described FIG. 7) (Block 1100):
acquisition of at least two 2D images showing the structures to be back-projected to 3D,
identification of these structures in the 2D images,
computation of the 3D position of the marked structures,
optional—visualization of the back-projected structures in the registered 3D context, and
overlay of the 3D back-projected structures to live 2D fluoro images

6. “Do Intervention” by performing several of the following steps in a loop (no specific order) (Block 1200):
turn the C-arm, choose zoom etc. to obtain an optimal working projection (registration follows).
acquire Fluoro images for X-ray control,
progress needle, catheter etc. to the target (fluoro guided),
adjust blending of 2D and 3D,
re-register 3D if patient movement has occurred,
verify device position in 3D under different C-arm angles etc., (registration follows); and

7. optional—verify success of the intervention by performing C-arm CT (Block 1300).

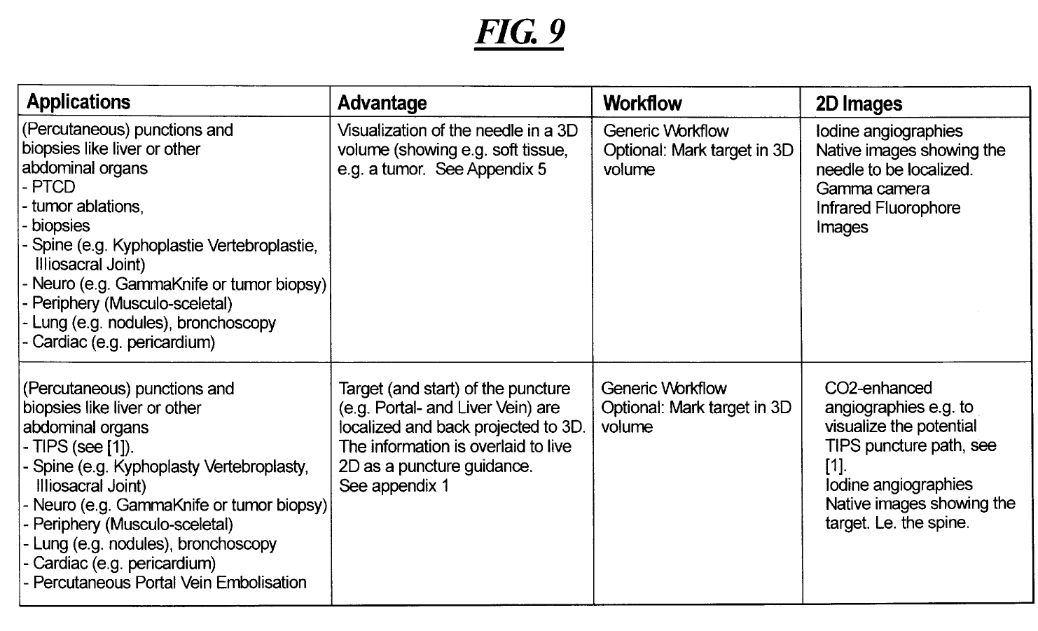

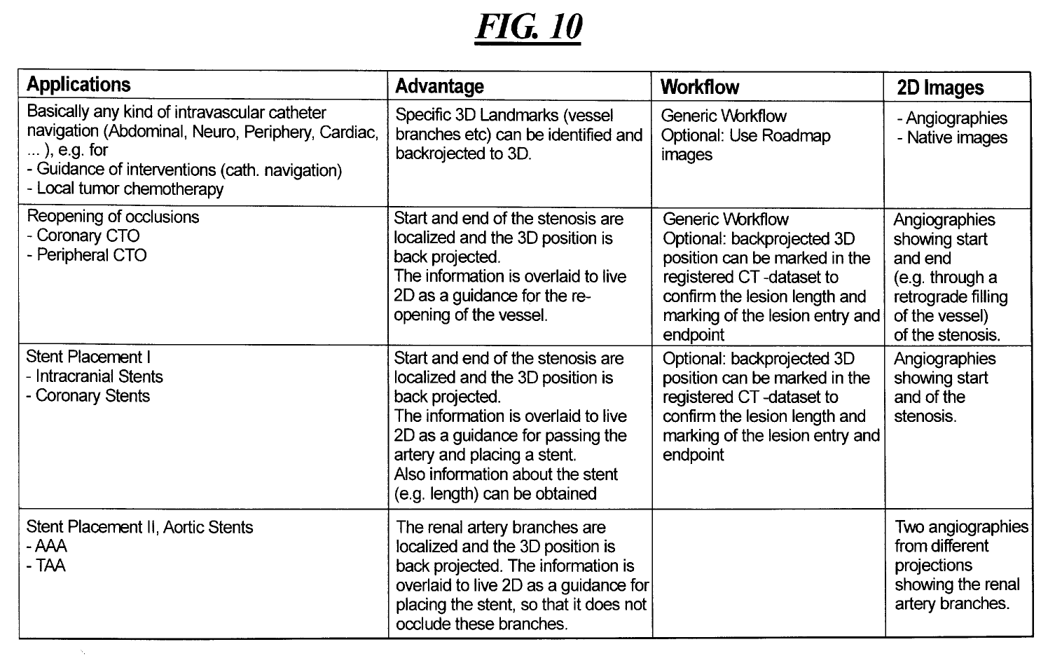

Example Applications are shown in the following tables. The FIG. 9 table shows needle or puncture guidance applications. The FIG. 10 table shows neurosurgery and vascular surgery or stent guidance applications. The FIG. 11 table shows neurosurgery and vascular surgery applications. And the FIG. 12 table shows spine procedures applications.

While preferred embodiments have been illustrated and described in detail in the drawings and foregoing description, the same are to be considered as illustrative and not restrictive in character, it being understood that only the preferred embodiments have been shown and described and that all changes and modifications that come within the spirit of the invention both now or in the future are desired to be protected.

We claim as our invention:

1. A method for performing an image-assisted medical procedure involving placing a medical device in a 3D human subject, comprising the steps of:
obtaining at least a first and a second 2D projected images of the 3D subject at a respective first angle and at a respective second angle, said 3D subject having at least one of the points selected from the group consisting of a start point and an end point for guidance of said medical device;
identifying at least one of said start point and said end point in each of the first and second 2D images;
calculating 3D positions of at least one of the start point and the end point;
overlaying at least one of said start and end point 3D positions as a back-projection to a live 2D image; and
performing the procedure utilizing the live 2D image with at least one of the start and end points as a guide to place said medical device.

2. The method of claim 1 wherein said medical device comprises a wire.

3. The method of claim 1 wherein said medical device comprises a needle.

4. The method of claim 1 wherein said medical device comprises a wire, both said start point and said end point are identified, and said wire is guided from said start point to said end point.

5. The method of claim 1 wherein said medical device comprises a needle, both said start point and said end point are identified, and said needle is guided from said start point to said end point.

6. The method of claim 1 wherein the live 2D image comprises a live 2D fluoro image.

7. The method of claim 1 including the step of calculating said 3D positions of at least one of the start point and the end point based on at least one of the identified start and end points in each of the first and second 2D images and the first and second angles.

8. The method of claim 1 wherein both said start and end points are identified, and a 3D line is calculated between the start and end points.

9. The method of claim 1 wherein said live 2D image includes a recalculated 3D position of at least one of the start and the end points.

10. The method of claim 1 wherein said medical device comprises a wire and said medical procedure involves placing said wire in said 3D human subject to open up a stenosis.

11. The method of claim 10 wherein said stenosis comprises a chronic total occlusion blood vessel.

12. The method of claim 1 wherein said medical device comprises a wire and said medical procedure comprises placing said wire as a guide wire in said 3D human subject to guide a stenting.

13. The method of claim 12 wherein said stenting comprises an intracranial stenting.

14. The method of claim 12 wherein said stenting comprises stenting of an abdominal aortic aneurysm.

15. The method according to claim 1, wherein the step of identifying at least one of the start point and the end point comprises manually selecting the at least one point on an image display with a pointing device.

16. The method according to claim 1, further comprising providing a 3D dataset of a feature of the subject via at least one of: a) a preoperative imaging selected from the group consisting of CT and MR; and b) an intra operative procedure selected from the group consisting of a C-arm CT and 3D Angio.

17. The method according to claim 16, wherein the 3D dataset is registered in a C-arm system used for the imaging.

18. The method according to claim 1 wherein the further projected 2D image comprises a feature of the subject where the medical device placement procedure is being performed.

19. The method of claim 1 further comprising tracking a position of the wire within the subject during the subject procedure.

20. The method of claim 1 further comprising:
providing a 3D dataset of a feature of the subject, and tracking the position of the medical device within the 3D dataset of the subject feature.

21. The method of claim 16 wherein the position of the medical device is tracked with a position sensor.

22. The method of claim 1, wherein the subject procedure utilizes a C-arm angio system.

23. A system for performing an image-assisted medical procedure involving placing a medical device in a 3D human subject, comprising:
an imaging system comprising an image acquisition device for acquiring an image of said 3D human subject;
a processor used to process acquired images;
a memory for storing acquired images and processed images;
an orientation mechanism to orient the imaging system to capture first and second 2D images of the 3D human subject at a respective first angle and at a respective second angle;
a display for providing the first and second 2D images to a user;
a selection unit via which the user can select at least one of the points selected from the group consisting of a start point and an end point to be used for placement of said medical device in said first and second 2D projected images;
a software module that calculates 3D positions of at least one of the start point and the end point;
a display upon which a live 2D image is shown; and
said software module overlaying said 3D positions of at least one of the start and end points as a back-projection to said live 2D image so that the user can perform the procedure using the live 2D image with at least one of the start and end points as a guide to place said medical device.

24. The system of claim 23 wherein said medical device comprises a wire.

25. The system of claim 23 wherein said medical device comprises a needle.

26. The system of claim 23 wherein said medical device comprises a wire and said wire is guided from said start point to said end point.

27. The system of claim 23 wherein said medical device comprises a needle and said needle is guided from said start point to said end point.

28. The system of claim 23 wherein the medical device comprises a wire and the medical procedure involves placing said wire in said 3D human subject to open up a stenosis.

29. The system of claim 23 wherein the medical device comprises a wire and said medical procedure comprises placing said wire as a guide wire in said 3D human subject to guide a stenting.
